(19) United States
(12) Patent Application Publication
(10) Pub. No.: US 2020/0024638 A1
(43) Pub. Date: Jan. 23, 2020

(54) BIOLOGICAL SAMPLE AND BIOLOGICAL INSTRUMENT CLEANLINESS MEASUREMENT KIT AND METHOD

(71) Applicant: KIKKOMAN CORPORATION, Chiba (JP)

(72) Inventors: Yuki TSUKADA, Chiba (JP); Shigeya SUZUKI, Chiba (JP); Yuko ICHIYANAGI, Chiba (JP)

(73) Assignee: KIKKOMAN CORPORATION, Chiba (JP)

(21) Appl. No.: 16/483,574

(22) PCT Filed: Feb. 9, 2018
(86) PCT No.: PCT/JP2018/004721

§ 371 Date: Aug. 5, 2019

(30) Foreign Application Priority Data
Feb. 9, 2017 (JP) 2017-022020
Mar. 24, 2017 (JP) 2017-059008
Aug. 14, 2017 (JP) 2017-156631
Oct. 2, 2017 (JP) 2017-192752

(51) Int. Cl. C12Q 1/66; C12Q 1/48; C12N 9/12; G01N 21/76

(52) U.S. Cl. CPC C12Q 1/66; C12Q 1/485; G01N 21/76; C12Y 207/01001; C12N 9/1205

(57) ABSTRACT

Provided are a kit and a method for measuring cleanliness of a bio-related sample and a bio-related instrument, which is not susceptible to ATP-degrading activity. Provided are a method for measuring cleanliness of a bio-related sample or a bio-related instrument and a kit therefor, comprising using an enzyme that catalyzes a reaction that produces ATP from ADP, luciferin, luciferase, and a metal salt. Also provided are a method further comprising using pyruvate orthophosphate dikinase (PPDK), adenylate kinase (ADK), or pyruvate-water dikinase (PWDK) and a kit further comprising PPDK, ADK, or PWDK. Further provided are a method for measuring cleanliness of a bio-related sample or a bio-related instrument and a kit therefor, comprising using an enzyme that catalyzes a reaction that produces ATP from AMP, an enzyme that catalyzes a reaction that produces AMP from ADP, luciferin, luciferase, and a metal salt.

### TECHNICAL FIELD

The present invention relates to a kit and a method for measuring cleanliness of a bio-related sample and a bio-related instrument, for example, a kit and a method for measuring cleanliness of a blood-related sample and a blood-related instrument.

### BACKGROUND ART

Instruments and environment handling bio-related samples are required to be clean since biogenic substances (liquids or solids) can be a cause of infection with pathogenic bacteria and viruses. In particular, the handling of blood in the healthcare setting needs to be careful since, for example, it causes viral infection. Moreover, it is important for the safety of patients and medical personnel that blood-related instruments and medical instruments such as surgical instruments, operating tables, endoscopes, clothes, and gloves as well as environments such as beds, bed fences, doorknobs, switches, and nurse call buttons are kept clean.

A method for measuring a substance characteristic of biogenic substances is used to examine whether biogenic substances are attached to or remain in a bio-related sample, a bio-related instrument, or an environment. For example, a method for measuring a substance characteristic of blood is used to examine whether blood is attached to or remains in a blood-related instrument, a medical instrument, or an environment. Biogenic substances are known to contain adenosine triphosphate (hereinafter, referred to as ATP) and in particular, blood is known to contain a large quantity of ATP.

Known representative methods for measuring ATP include a method comprising causing a reaction of ATP and the substrate, luciferin in the presence of luciferase and measuring luminescence (Non Patent Literature 1). This reaction is catalyzed by luciferase and occurs in the presence of a divalent metal ion as follows.

Luciferin +ATP+O₂→oxyluciferin+adenosine monophosphate (AMP)+pyrophosphoric acid (PPi)+CO₂+light

Luciferase is found in bacteria, protozoans, mollusks, insects, and the like. Examples of the insects having luciferase include beetles, for example, firefly and click beetle. Many genes for luciferase have been isolated and their nucleotide sequences have been also determined.

Patent Literature 1 describes a method for measuring ATP in a blood sample. Patent Literature 2 describes a method for examining cleanliness comprising measuring ATP, adenosine monophosphate (AMP), and adenosine diphosphate (ADP). This method uses pyruvate orthophosphate dikinase (PPDK), phosphoenolpyruvic acid (PEP), pyrophosphoric acid (PPi), luciferin, luciferase, and a metal salt as well as pyruvate kinase (PK). Samples to be measured are yeast extract, beef extract, malt extract, beer, bovine milk, rice, and pork.

Patent Literature 3 describes a method of judging fatigue. In Example, trichloroacetic acid (TCA), which is a protein denaturant, is added to collected whole blood, plasma, and erythrocytes to deactivate ATPase and, immediately after that, the amounts of ADP, ATP, and AMP contained in the samples are measured.

Patent Literature 4 describes PPDK and a method for producing the same. Patent Literature 5 describes a method for measuring ATP and AMP.

### CITATION LIST

### Patent Literature

Patent Literature 1: JP Patent Publication (Kokai) No. 2015-042156 A
Patent Literature 2: JP Patent Publication (Kokai) No. 11-69997 A (1999)
Patent Literature 3: International Publication No. WO 2005/012903
Patent Literature 4: JP Patent Publication (Kokai) No. 8-168375 A (1996) (JP Patent No. 3181801)
Patent Literature 5: JP Patent Publication (Kokai) No. 9-234099 A (1997) (JP Patent No. 3409962)

### Non Patent Literature

Non Patent Literature 1: Marlene DeLuca, William D. McElroy, Biochemistry, 1974, 13 (5), pp 921-925

### SUMMARY OF INVENTION

### Technical Problem

The present inventors measured residual ATP in diluted blood samples to examine how ATP contained in a blood-related sample is degraded over time and found that the initial residual ATP, which is 100%, is degraded to approximately 5 to 10% in several tens of minutes at 25° C. More specifically, the present inventors found that the measurement of ATP alone cannot accurately evaluate blood remaining in or attached to a blood-related sample. Thus, an object of the present invention is to provide a method for accurately detecting blood-derived contamination, which method is not susceptible to ATP-degrading activity.

Furthermore, the present inventors measured, using samples heated for different periods of time, how residual ATP changes over time in samples in which ATP may have been degraded and found that initial residual ATP, which is 100%, is degraded to approximately 50% in 120 hours at 80° C. More specifically, the present inventors found that the measurement of ATP alone cannot accurately evaluate remaining or attached biogenic substances in which ATP may have been degraded. Thus, an object of the present invention is to provide a method for accurately detecting biogenic contamination, which method is not susceptible to ATP-degrading activity.

### Solution to Problem

The measurement of ATP alone cannot accurately evaluate blood remaining in or attached to a blood-related instrument or the like. Moreover, the measurement of ATP alone cannot accurately evaluate biogenic substances remaining in or attached to a bio-related instruments or the like relating to biogenic substances in which ATP may have been degraded. Therefore, the present inventors then examined how ATP+ADP or ATP+ADP+AMP is degraded in diluted blood samples over time and found that the initial amount, which is 100%, is surprisingly maintained around 90 to 95% (ATP+ADP) or almost 100% (ATP+ADP+AMP) even for several tens of minutes at 25° C. Thus, the present inventors have found that the accurate detection of blood remaining in or attached to blood-related instruments or the like is enabled by the measurement of ATP and ADP or ATP, ADP and AMP, but not ATP alone, thereby completing the present invention.

Moreover, in another embodiment, the present inventors examined how ATP+ADP or ATP+ADP+AMP is degraded over time in samples heated for a long time and found that the initial amount, which is 100%, is surprisingly maintained around 70 to 95% (ATP+ADP) or 90 to 100% (ATP+ADP+AMP) even after 8 hours at 80° C. Moreover, the present inventors compared the change over time of ATP+AMP with the change over time of ATP+ADP in heated samples and surprisingly found that ATP+ADP is more stable than ATP+AMP. Thus, the present inventors have found that more accurate detection of biogenic substances remaining in or attached to bio-related instruments or the like after long-time heating is enabled by the measurement of ATP and ADP or ATP, ADP and AMP, but not ATP alone, thereby completing the present invention.

The present invention includes the following embodiments:
[1] A kit for measuring cleanliness of a blood-related sample or a blood-related instrument, comprising an enzyme that catalyzes a reaction that produces ATP from ADP, luciferin, luciferase, and a metal salt.
[2] A kit for measuring cleanliness of a bio-related sample or a bio-related instrument, comprising an enzyme that catalyzes a reaction that produces ATP from ADP, luciferin, luciferase, and a metal salt.
[3] The kit according to 1 or 2, wherein the enzyme that catalyzes a reaction that produces ATP from ADP is selected from the group consisting of pyruvate kinase (PK), acetate kinase (AK), creatine kinase (CK), polyphosphate kinase (PPK), hexokinase, glucokinase, glycerol kinase, fructokinase, phosphofructokinase, riboflavin kinase, and fructose-bisphosphatase.
[4] The kit according to any of 1 to 3, further comprising an enzyme that catalyzes a reaction that produces ADP or ATP from AMP.
[5] The kit according to 4, wherein the enzyme that catalyzes a reaction that produces ADP or ATP from AMP is pyruvate orthophosphate dikinase (PPDK), adenylate kinase (ADK), or pyruvate-water dikinase (PWDK).
[6] A kit for measuring cleanliness of a blood-related sample or a blood-related instrument, comprising an enzyme that catalyzes a reaction that produces ATP from AMP, an enzyme that catalyzes a reaction that produces AMP from ADP, luciferin, luciferase, and a metal salt.
[7] A kit for measuring cleanliness of a bio-related sample or a bio-related instrument, comprising an enzyme that catalyzes a reaction that produces ATP from AMP, an enzyme that catalyzes a reaction that produces AMP from ADP, luciferin, luciferase, and a metal salt.
[8] The kit according to 6 or 7, wherein the enzyme that catalyzes a reaction that produces ATP from AMP is pyruvate orthophosphate dikinase (PPDK) or pyruvate-water dikinase (PWDK), and wherein the enzyme that catalyzes a reaction that produces AMP from ADP is ADP-dependent hexokinase or apyrase.

[9] The kit according to any of 1, 3 to 6, and 8, wherein the blood-related sample is a sample to or in which blood may be attached or remain, or the blood-related instrument is an instrument to or in which blood may be attached or remain.
[10] The kit according to any of 2 to 5 and 7 to 8, wherein the bio-related sample is a sample to or in which a biogenic substance may be attached or remain, wherein ATP contained in the biogenic substance may have been degraded, or the bio-related instrument is an instrument to or in which a biogenic substance may be attached or remain, wherein ATP contained in the biogenic substance may have been degraded.
[11] The kit according to 10, wherein the bio-related sample is a sample to or in which sweat may be attached or remain, or the bio-related instrument is an instrument to or in which sweat may be attached or remain.
[12] The kit according to any of 1 to 8, wherein the blood-related instrument or bio-related instrument is an endoscope.
[13] A method for measuring cleanliness of a blood-related sample or a blood-related instrument, comprising using an enzyme that catalyzes a reaction that produces ATP from ADP, luciferin, luciferase, and a metal salt.
[14] A method for measuring cleanliness of a bio-related sample or a bio-related instrument, comprising using an enzyme that catalyzes a reaction that produces ATP from ADP, luciferin, luciferase, and a metal salt.
[15] The method according to 13 or 14, wherein the enzyme that catalyzes a reaction that produces ATP from ADP is selected from the group consisting of pyruvate kinase (PK), acetate kinase (AK), creatine kinase (CK), polyphosphate kinase (PPK), hexokinase, glucokinase, glycerol kinase, fructokinase, phosphofructokinase, riboflavin kinase, and fructose-bisphosphatase.
[16] The method according to any of 13 to 15, further comprising an enzyme that catalyzes a reaction that produces ADP or ATP from AMP.
[17] The method according to 16, wherein the enzyme that catalyzes a reaction that produces ADP or ATP from AMP is pyruvate orthophosphate dikinase (PPDK), adenylate kinase (ADK), or pyruvate-water dikinase (PWDK).
[18] A method for measuring cleanliness of a blood-related sample or a blood-related instrument, comprising using an enzyme that catalyzes a reaction that produces ATP from AMP, an enzyme that catalyzes a reaction that produces AMP from ADP, luciferin, luciferase, and a metal salt.
[19] A method for measuring cleanliness of a bio-related sample or a bio-related instrument, comprising using an enzyme that catalyzes a reaction that produces ATP from AMP, an enzyme that catalyzes a reaction that produces AMP from ADP, luciferin, luciferase, and a metal salt.
[20] The method according to 18 or 19, wherein the enzyme that catalyzes a reaction that produces ATP from AMP is pyruvate orthophosphate dikinase (PPDK) or pyruvate-water dikinase (PWDK), and wherein the enzyme that catalyzes a reaction that produces AMP from ADP is ADP-dependent hexokinase or apyrase.
[21] The method according to any of 13, and 15 to 18, and 20, wherein the blood-related sample is a sample to or in which blood may be attached or remain, or the blood-related instrument is an instrument to or in which blood may be attached or remain.
[22] The method according to any of 14 to 17 and 19 to 20, wherein the bio-related sample is a sample to or in which a biogenic substance may be attached or remain, wherein ATP contained in the biogenic substance may have been degraded, or the bio-related instrument is an instrument to or in which a biogenic substance may be attached or remain, wherein ATP contained in the biogenic substance may have been degraded.
[23] The method according to 22, wherein the bio-related sample is a sample to or in which sweat may be attached or remain, or the bio-related instrument is an instrument to or in which sweat may be attached or remain.
[24] The method according to any of 13 to 20, wherein the blood-related instrument or a bio-related instrument is an endoscope.

The present specification encompasses the contents disclosed in Japanese Patent Application Nos. 2017-022020, 2017-059008, 2017-156631, and 2017-192752, to which the present application claims priority.

### Advantageous Effects of Invention

According to the present invention, ATP, AMP, ADP derived from biogenic substances, for example, blood can be measured even in samples in which ATP has been degraded by heat, pH, time progress, or ATPase. Therefore, the cleanliness of bio-related samples and bio-related instruments can be measured as an effect of the present invention. Moreover, the cleanliness of blood-related samples and blood-related instruments can be measured. Moreover, environments to or in which a biogenic substance, for example, blood or a solid is suspected to be attached or remain can be examined, and the cleanliness can be measured. Moreover, the cleanliness of samples and instruments after long-time heating can be measured.

For example, surgical instruments and endoscopes are sometimes washed after having been soaked in a washing tank or a lavage fluid for a certain time. Contamination may not be detected if only ATP is measured for the surgical instrument or endoscope after the washing. However, it does not necessarily mean that the instrument is clean, but contamination may remain according to the findings of the present invention. The method of the present invention makes it possible to measure the cleanliness more accurately by measuring ATP+ADP or ATP+ADP+AMP, for example, on a medical instrument or an endoscope after the washing, than by measuring ATP alone.

Moreover, washing steps for bio-related instruments may also include a heating step and/or a drying step. If only ATP on the instrument after heating or drying is measured, contamination may not be detected. However, it does not necessarily mean that the instrument is clean, but contamination may remain according to the findings of the present invention. The method of the present invention makes it possible to measure the cleanliness of for example, an instrument after heating more accurately by measuring ATP+ADP or ATP+ADP+AMP than by measuring ATP alone.

### BRIEF DESCRIPTION OF DRAWINGS

FIG. 1 illustrates change of luminescence level over time when a blood sample is diluted 50 times and stored at 25° C.

FIG. 2-1 illustrates a result of examination of ATP degradation in a heated sample over time at pH 4 (10 hours).

FIG. 2-2 illustrates a result of examination of ATP degradation in a heated sample over time at pH 4 (120 hours).

FIG. 3-1 illustrates a result of examination of ATP degradation in a heated sample over time at pH 7 (10 hours).

FIG. 3-2 illustrates a result of examination of ATP degradation in a heated sample over time at pH 7 (120 hours).

FIG. 4-1 illustrates a result of examination of ATP degradation in a heated sample over time at pH 11 (10 hours).

FIG. 4-2 illustrates a result of examination of ATP degradation in a heated sample over time at pH 11 (120 hours).

FIG. 5 is a standard curve.

FIG. 6 is a standard curve.

FIG. 7 illustrates change of luminescence level over time when a saliva sample is diluted 200 times and stored.

FIG. 8 illustrates change of luminescence level over time when a sample obtained by suspending a cotton swab used to wipe an endoscope is stored at 25° C.

FIG. 9-1 is a standard curve.

FIG. 9-2 is a standard curve.

FIG. 9-3 is a standard curve.

FIG. 10-1 is a standard curve.

FIG. 10-2 is a standard curve.

FIG. 10-3 is a standard curve.

### DESCRIPTION OF EMBODIMENTS

In a certain embodiment, the present invention provides a method for measuring cleanliness of a bio-related sample or a bio-related instrument. In another embodiment, the present invention provides a method for measuring cleanliness of a blood-related sample or a blood-related instrument. In a certain embodiment, the method of the present invention comprises using an enzyme that catalyzes a reaction that produces ATP from ADP, luciferin, luciferase, and a metal salt. The enzyme that catalyzes a reaction that produces ATP from the ADP may be selected from the group consisting of pyruvate kinase (PK), acetate kinase (AK), creatine kinase (CK), polyphosphate kinase (PPK), hexokinase, glucokinase, glycerol kinase, fructokinase, phosphofructokinase, riboflavin kinase, and fructose-bisphosphatase. In another embodiment, the method of the present invention further comprises using pyruvate orthophosphate dikinase (PPDK), adenylate kinase or pyruvate-water dikinase (PWDK).

Moreover, in a certain embodiment, the present invention provides a kit for use in measuring cleanliness of a bio-related sample or a bio-related instrument. In another embodiment, the present invention provides a kit for use in measuring cleanliness of a blood-related sample or a blood-related instrument. The kit of the present invention comprises an enzyme that catalyzes a reaction that produces ATP from ADP, luciferin, luciferase, and a metal salt, and optionally an instruction. The enzyme that catalyzes a reaction that produces ATP from ADP may be selected from the group consisting of pyruvate kinase (PK), acetate kinase (AK), creatine kinase (CK), polyphosphate kinase (PPK), hexokinase, glucokinase, glycerol kinase, fructokinase, phosphofructokinase, riboflavin kinase, and fructose-bisphosphatase. In another embodiment, the kit of the present invention further comprises pyruvate orthophosphate dikinase (PPDK), adenylate kinase or pyruvate-water dikinase (PWDK).

If the sample contains ATP, this is converted into AMP by luciferase and luminescence is produced. If the sample contains ADP, this is converted into ATP by an enzyme that catalyzes a reaction that produces ATP from ADP and ATP is then subjected to the luminescent reaction. In this way, the total amount of ATP and ADP present in a system can be measured. Furthermore, if the sample contains AMP in a system where PPDK is present, this is converted into ATP by PPDK, PEP, and PPi. Alternatively, if the sample contains AMP in a system where PWDK is present, this is converted into ATP by PWDK, PEP, and phosphoric acid. Produced ATP causes luminescence by luciferase again. Since the luminescence is stably maintained and the level of luminescence correlates with the total amount of ATP and AMP present in the system, the quantification of ATP and AMP is possible. If an enzyme that catalyzes a reaction that produces ATP from ADP and PPDK, ADK or PWDK are present, the total amount of ATP, ADP, and AMP can be measured. The advantage of the method using PPDK or the like is that the luminescence can be stably measured even with an apparatus with low sensitivity without attenuation of a level of luminescence because AMP produced by luciferase is also converted into ATP.

If the sample contains AMP and ATP, these are converted into 2 molecules of ADP by adenylate kinase. The produced ADP molecules can then be converted into ATP by the enzyme that catalyzes a reaction that produces ATP from ADP. The produced ATP can then be detected by luciferase.

According to the findings that the present inventors have found, the measurement of ATP alone may be unable to detect contamination accurately for biogenic substances. Biogenic substances contain ATP, and ATP can be relatively easily dephosphorylated into ADP. Moreover, according to the findings that the present inventors have found, ADP is converted into AMP only when it is stored at a high temperature for a long time. ADP can also finally turn into AMP, but according to the findings of the present inventors, the phosphate in AMP derived from a biogenic substance is not easily dephosphorylated into adenosine. Therefore, the measurement of the 2 components ATP and ADP or the 3 components ATP, ADP, and AMP allows a stable measurement of ATP (or degradation products thereof) contained in a biogenic substance.

Moreover, the blood contains ATPases and ADP degrading enzymes, but according to the findings that the present inventors have found, the measurement of the 2 components ATP and ADP or the 3 components ATP, ADP, and AMP allows a stable measurement of ATP (or degradation products thereof) contained in the blood. Although not wishing to be bound by a particular theory, this is considered to be because the enzymatic activity to convert ADP into AMP is weak and/or enzymes that can dephosphorylate and degrade AMP into adenosine are limited or the activity of such enzymes is weak. Therefore, the measurement of the 2 components ATP and ADP or the 3 components ATP, ADP, and AMP allows stable measurement of even samples left, without influence of the degradation activity and the timing of measurement, and without missing uncleanness, and is excellent as an indicator in the examination of cleanliness.

### [Luciferase]

In a certain embodiment, the kit of the present invention comprises luciferase and luciferin. In this case, a metal ion such as magnesium, manganese, or calcium may also be contained. Those skilled in the art can determine the concentration of the metal ion depending on the enzyme to be used. By luciferase required, ATP, O₂, and luciferin are converted into AMP, pyrophosphoric acid, CO₂, and oxyluciferin, and luminescence is generated at this time. The luciferase may be natural luciferase or may be a genetically engineered recombinant luciferase mutant. The luciferase mutant may be generated by site-directed mutagenesis or by random mutagenesis. The luciferase mutant may be a fusion protein with a protein having another function. The luciferase mutant may have desired properties such as improved heat resistance, or improved surfactant resistance.

The level of luminescence from luciferase can be evaluated using relative luminescence intensity (RLU) as an indicator obtained using an appropriate apparatus for measuring luminescence, for example, a luminometer (CentroLB960 or Lumat3 LB9508 manufactured by Berthold Technologies GmbH & Co. KG; Lumitester C-110, Lumitester C-100, Lumitester PD-20, Lumitester PD-30 manufactured by Kikkoman Biochemifa Company, or the like). Typically, luminescence generated in conversion from luciferin to oxyluciferin is measured. As apparatuses for measuring luminescence, apparatuses with a photomultiplier tube (those manufactured by 3M Corporation, and the like) and apparatuses with a photodiode (those manufactured by Hygiena, LLC, Neogen Corporation, and the like) capable of high sensitivity measurement can also be used.

The luciferase is not particularly limited, as long as its substrate is ATP, but those derived from bacteria, protozoans, animals, mollusks, and insects can be used. Examples of those derived from insects include coleopteran luciferase, and examples thereof include those derived from fireflies such as the genus Photinus, for example, Photinus pyralis; the genus Photuris, for example, Photuris lucicrescens, Photuris pennsylvanica; the genus Luciola, for example, Luciola cruciata, Luciola lateralis, Luciola parvula; the genus Pyrocoelia; and Lucidina biplagiata; and those derived from click beetle in the genus Pyrophorus. Many genes for luciferase have been reported, and their nucleotide sequences and amino acid sequences are available from known databases such as GeneBank.

The luciferase gene may be a wildtype gene or a gene having mutation. The mutation may be a mutation introduced site-specifically or a random mutation. Examples of known mutations include, but are not limited to, mutations that improve the level of luminescence as described in JP Patent Publication (Kokai) No. 2011-188787 A; mutations that increase the luminescence durability as described in JP Patent Publication (Kokai) No. 2000-197484 A; mutations that change the luminescence wavelength as described in JP Patent No. 2666561 or JP Patent Publication (Kohyo) No. 2003-512071 A; mutations that increase resistance to surfactant as described in JP Patent Publication (Kokai) No. 11-239493 A (1999); mutations that increase affinity for substrate as described in International Publication No. WO 99/02697 pamphlet, JP Patent Publication (Kohyo) No. 10-512750 A (1998), or JP Patent Publication (Kohyo) No. 2001-518799 A; and mutations that increase the stability as described in JP Patent No. 3048466, JP Patent Publication (Kokai) No. 2000-197487 A, JP Patent Publication (Kohyo) No. 9-510610 A (1997), and JP Patent Publication (Kohyo) No. 2003-518912 A.

The luciferase gene and a recombinant DNA thereof can be prepared in a conventional way. For example, JP Patent Publication (Kokoku) No. 7-112434 B (1995) describes a luciferase gene from Luciola lateralis. Moreover, JP Patent Publication (Kokai) No. 1-51086 A (1989) describes a luciferase gene from Luciola cruciata.

The luciferase gene can be incorporated into a vector such as a plasmid, a bacteriophage, or a cosmid, with which an appropriate host may be transformed or transfected. The host may be a microorganism, a bacterium such as Escherichia coli, yeast, or the like. The transformed host having luciferase-producing ability can be cultured by various known methods.

Examples of the medium include those obtained by adding, to one or more nitrogen sources such as triptone, yeast extract, meat extract, peptone, corn steep liquor, or an exudate of soybean or wheat bran, one or more inorganic salts such as sodium chloride, potassium dihydrogen phosphate, dipotassium phosphate, magnesium chloride, ferric chloride, magnesium sulfate, or manganese sulfate, and as necessary carbohydrate raw materials, vitamins, and the like.

The initial pH of the medium can be, for example, 7 to 9. The culture can be conducted, for example, at 30 to 40° C. for 2 to 24 hours, by aerated and agitated culture, shaking culture, static culture, or the like. After culturing, the luciferase is collected from the culture by a known technique.

Specifically, luciferase is extracted by subjecting the cells to an ultrasonic homogenization treatment, a grinding treatment, or the like by a conventional way or using a lytic enzyme such as lysozyme. A crude enzyme can be obtained by treating the resultant extract by filtration, centrifugation, or the like, removing nucleic acid by streptomycin sulfate or the like as needed, and adding ammonium sulfate, alcohol, acetone, or the like to fractionate this.

The crude enzyme may further be purified by various techniques such as gel filtration and chromatography. Commercially available luciferase can also be used and, for example, the luciferase of Kikkoman Biochemifa Company, cat. No. 61314 can be used. This luciferase is a luciferase described in JP Patent Publication (Kokai) No. 11-239493 A (1999) (JP Patent No. 3749628) (SEQ ID NO: 1 in the literature). Moreover, commercially available luciferase from Sigma Aldrich, Promega KK., Molecular Probes (R) of Life Technology Inc. can also be used.

### [Luciferin]

The luciferin may be any one as long as it is recognized as a substrate by the luciferase to be used and may be natural or chemically synthesized. Moreover, any known luciferin derivative may also be used. The basic structure of luciferin is imidazopyrazinone, and there are many tautomers thereof. Examples of the luciferin include firefly luciferin. The firefly luciferin is a substrate of firefly luciferase (EC 1.13.12.7). The luciferin derivative may be those described in JP Patent Publication (Kokai) No. 2007-91695 A, JP Patent Publication (Kohyo) No. 2010-523149 A (International Publication No. 2008/127677).

In a certain embodiment, the final concentration (mg protein/mL) of luciferase in the system of measurement can be 0.001 mg protein/mL or more, 0.01 mg protein/mL or more, 0.02 mg protein/mL or more, 0.05 mg protein/mL or more, 0.10 mg protein/mL or more, 0.20 mg protein/mL or more, or 0.25 mg protein/mL or more when measuring luciferase concentration by absorbance at 280 nm. In a certain embodiment, the final concentration (mg protein/mL) in the system of measurement of luciferase can be 1 mg protein/mL or less, 0.5 mg protein/mL or less, or 0.3 mg protein/mL or less when measuring luciferase concentration by absorbance at 280 nm. In a certain embodiment, the final concentration in the system of measurement of luciferin or a luciferin derivative can be 0.01 mM to 20 mM, 0.05 mM to 20 mM, 0.1 mM to 20 mM, 0.5 mM to 10 mM, for example, 0.75 mM to 5 mM.

Enzyme that Catalyzes a Reaction that Produces ATP from ADP

In a certain embodiment, the method of the present invention comprises using an enzyme that catalyzes a reaction that produces ATP from ADP. By the enzyme that catalyzes a reaction that produces ATP from ADP, ADP present in the system is converted into ATP. Then, ATP is converted into AMP by luciferase and luminescence is produced along with the conversion.

As the enzyme that catalyzes a reaction that produces ATP from ADP, any known enzyme can be used, and examples thereof include kinases having ATP-producing ability. Examples of the kinases having ATP-producing ability include, but are not limited to, pyruvate kinase, acetate kinase, creatine kinase, polyphosphate kinase, hexokinase, glucokinase, glycerol kinase, fructokinase, phosphofructokinase, riboflavin kinase, fructose-bisphosphatase and combinations thereof.

### [Pyruvate Kinase (PK)]

The pyruvate kinase (EC 2.7.1.40) converts phosphoenolpyruvate into pyruvate in glycolysis, and ADP is converted into ATP at the same time. This reaction is an exergonic reaction, where the change in the Gibbs energy is negative, and irreversible under natural conditions:

### PEP +ADP Pyruvate+ATP

The reverse reaction is catalyzed by pyruvate carboxylase and phosphoenolpyruvate carboxykinase in gluconeogenesis and produces PEP and ADP from ATP and pyruvic acid. When cell extraction is performed, various enzymes are mixed in the system and the aforementioned reactions can progress in the both directions. In the system, if the phosphoenolpyruvate is present at a high concentration, ADP can be converted into ATP. Moreover, it is considered that if not only phosphoenolpyruvate, but also pyruvate kinase is present in the system, more ADP is converted into ATP. The pyruvate kinase is not particularly limited, but, for example, those derived from animals such as rabbit, rat, and chicken and microorganisms such as yeast and Bacillus stearothermophilus can be used.

### [Acetate Kinase (AK)]

The acetate kinase (EC 2.7.2.1) catalyzes conversion from ATP and acetate to ADP and acetylated phosphate and vice versa in the presence of a cation: ATP+acetate←→ADP+acetylated phosphate

The acetate kinase (AK) is also referred to as ATP:acetate phosphotransferase or acetyl kinase. As used herein, these terms are exchangeable to each other. In the living body, ADP and acetylated phosphate are produced from ATP and acetate, and the reactions to produce acetyl CoA are finally promoted. If acetylated phosphate and ADP produced from acetyl CoA are present in the system, these can be converted into acetate and ATP. The acetate kinase is not particularly limited, but those derived from microorganisms such as Escherichia coli, Bacillus stearothermophilus, Costridium pasteurianum, Lactobacillus delbruckii, and Veillonella alcalescence can be used.

### [Creatine Kinase (CK)]

The creatine kinase (EC 2.7.3.2) mediates the conversion reaction from creatine and ATP to creatine phosphate and ADP and vice versa: Creatine+ATP←→Creatine Phosphate +ADP

The creatine kinase (CK) is also referred to as creatine phosphokinase (CPK) or phosphocreatine kinase. As used herein, these terms are exchangeable to each other. In muscles and the like of animals, creatine phosphate and ADP are usually produced from creatine and ATP. However, this reaction is a reversible reaction, and if creatine phosphate and ADP are present in the system at high concentrations, the reaction may progress in the reverse direction to produce creatine and ATP. In the living body, cytoplasmic creatine kinase is composed of two subunits of B or M. Therefore, the 3 isozymes CK-MM, CK-BB, and CK-MB can be present depending on the combination of the subunits. The isozymic pattern differs depending on the tissue, but any combination is available in the present invention. The creatine kinase is not particularly limited, but those derived from animals can be used, and examples thereof include those derived from rabbit, chicken, cow, pig, carp, catfish, and frog.

### [Polyphosphate Kinase (PPK)]

The polyphosphate kinase (EC 2.7.4.1) catalyzes the reaction to convert polyphosphate (PolyPn) and ADP into polyphosphate (PolyPn-1) and ATP:

### ADP+PolyPn←→ATP+PolyPn-1

The polyphosphate kinase (PPK) is also referred to as ATP:polyphosphate phosphotransferase. As used herein, these terms are exchangeable to each other. PPK is involved in oxidative phosphorylation in the living body. If polyphosphate (n) and ADP are present in the system, these can be converted into polyphosphate (n-1) and ATP. The polyphosphate kinase is not particularly limited, but, for example, those derived from microorganisms such as Escherichia coli, yeast, and Corynebacterium xerosis can be used.

### [Riboflavin Kinase (FMNK)]

The riboflavin kinase (EC 2.7.1.26) is also described as FMNK and catalyzes the reaction to convert riboflavin and ATP into riboflavin phosphate (FMN) and ADP:

### ATP+riboflavin←→ADP+FMN

The riboflavin kinase belongs to ATP:riboflavin 5′-phosphotransferase (also referred to as flavokinase). The riboflavin kinase is not particularly limited, but, for example, those derived from microorganisms and animals can be used, and examples thereof include those derived from yeast, rat, and a bean (Phaseolus radiatus).

### [Phosphofructokinase 1 (PFK1)]

The phosphofructokinase 1 (EC 2.7.1.11) is also described as PFK1 and catalyzes the reaction to convert fructose-6-phosphate (Fru6P) and ATP into fructose-1,6-bisphosphate (Fru1,6-BP) and ADP:

### Fru6P+ATP←→Fru1,6-BP+ADP

The phosphofructokinase 1 belongs to phosphofructokinase. As used herein, phosphofructokinase 1 may be described as Fru-1,6BPK. The phosphofructokinase 1 is not particularly limited, but those derived from animals and microorganisms can be used, and examples of those derived from microorganisms include those derived from baker's yeast, brewer's yeast, Clostridium pasteurianum, Escherichia coli, and Bacillus licheniformis.

### [Fructose-Bisphosphatase (FBPase)]

The fructose-bisphosphatase (EC 3.1.3.11) is also described as FBPase and catalyzes the reaction to convert fructose-1,6-bisphosphate (Fru1,6-BP) and ADP into fructose-6-phosphate (Fru6P) and ATP:

### Fru1,6-BP+ADP←→Fru6P+ATP

The fructose-bisphosphatase may also be described as FBP, FBP1. The fructose-bisphosphatase is not particularly limited, but those derived from animals, plants, and microorganisms can be used, and examples thereof include those derived from rabbit and chicken.

### [Pyruvate-Phosphate Dikinase (PPDK)]

The pyruvate-phosphate dikinase (EC 2.7.9.1) catalyzes the reaction between ATP, pyruvate, and orthophosphate, and adenosine monophosphate (AMP), phosphoenolpyruvate (PEP), and pyrophosphate (PPi):

### ATP+pyruvate+phosphate←→AMP+PEP+PPi

The pyruvate-phosphate dikinase (PPDK) is also referred to as ATP:pyruvate, phosphate phosphotransferase, pyruvate orthophosphate dikinase, pyruvate phosphate ligase. As used herein, these terms are exchangeable to each other. PPDK usually converts pyruvate into PEP, and 1 molecule of ATP is consumed and converted into AMP in the process. The reaction is divided into the following 3 reversible reactions.
1. The enzyme PPDK binds to ATP, converts into AMP, and produces diphosphorylated PPDK.
2. The diphosphorylated PPDK binds to inorganic phosphate and produces diphosphate and monophosphorylated PPDK.
3. The monophosphorylated PPDK binds to pyruvate and produces PEP and reproduces PPDK.

In the reactions, if the concentration of PEP present in the system is high, reactions progress in the reverse direction as follows.

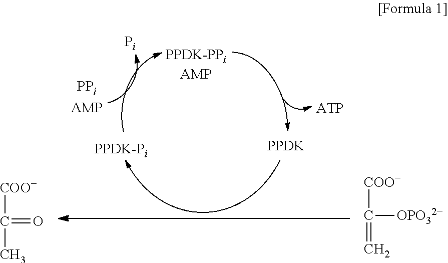

For convenience, the reaction steps are described with the same number as those described above.
3. The PEP binds to PPDK and produces monophosphorylated PPDK and pyruvate.
2. From the diphosphate and monophosphorylated PPDK, diphosphorylated PPDK and inorganic phosphate are produced.
1. From the diphosphorylated PPDK and AMP, PPDK and ATP are produced.

### [Adenylate Kinase (ADK)]

The adenylate kinase (EC 2.7.4.3) is also referred to as ADK and catalyzes the following reaction in the presence of a metal ion:

### ATP+AMP←→2ADP

This reaction is reversible. ADK is an example of the enzyme that catalyzes the reaction that produces ADP from AMP. By combining ADK with PK or the like, ADP is converted into ATP, and as a result, ATP and ADP and AMP can be measured.

### [Pyruvate-Water Dikinase (PWDK)]

The pyruvate-water dikinase (EC 2.7.9.2) catalyzes the following reaction: ATP+pyruvate+H₂O←→AMP+phosphoenolpyruvate (PEP)+phosphate (P)

The pyruvate-water dikinase is also referred to as phosphoenolpyruvate synthase; pyruvate-water dikinase (phosphorylation); PEP synthetase; phosphoenolpyruvate synthetase; phosphoenolpyruvic synthetase; and phosphopyruvate synthetase. As used herein, these terms are exchangeable to each other.

By using PWDK with PEP, the ATP production from AMP and PEP can be promoted. By combining PWDK with PK or the like, ADP is converted into ATP, and as a result, ATP and ADP and AMP can be measured.

### [RNase]

In a certain embodiment, the kit of the present invention may comprise RNase. Moreover, in a certain embodiment, the method of the present invention may comprise using RNase. The RNase here means an RNase not derived from the sample.

As used herein, the RNase means an enzyme that catalyzes the reaction that produces 5′-mononucleotide (AMP, GMP, CMP, and UMP) from RNA, and examples thereof include the following: (1) Endonuclease S₁ (EC3.1.30.1), (2) Venom exonuclease (EC3.1.15.1), (3) Phosphodiesterase 1 (EC3.1.4.1). The Endonuclease S₁ includes Nuclease P₁, Mung beans nuclease, and Neurospora crassa nuclease.

In another embodiment, the kit of the present invention does not comprise RNase or does not comprise a substantial amount of RNase. Moreover, in a certain embodiment, the method of the present invention does not comprise using RNase or does not comprise using a substantial amount of RNase. In this embodiment, RNases derived from the sample may be contained in the reaction system. As used herein, the “substantial amount of RNase” means an amount of RNase that does not affect the effect (for example, the effect of providing a method for accurate detection of contamination, that is not susceptible to the ATP-degrading activity) of the kit or method of the present invention. Examples of not comprising a substantial amount of RNase include a kit comprising RNase, for example, at a final concentration of 0.3 U/ml or less, 0.15 U/ml or less, 0.1 U/ml or less, 0.05 U/ml or less, 0.01 U/ml or less, or 0.001 U/l or less in the reaction system, or a method comprising using such an amount of RNase. As used herein, the enzyme unit of RNase is defined as an amount of enzyme having an activity unit (U) of the enzyme having the RNA-degrading ability that converts 1.0 μmole per minute of substrate into acid-soluble nucleotide at 37° C., in view of the RNA-degrading ability of the enzyme. For example, the enzyme unit of Nuclease P₁ is defined as an amount of enzyme that converts 1.0 μmole of substrate per 1 minute into acid-soluble nucleotide at pH 5.3 at 37° C. (for detail on the definition of the enzyme activity of Nuclease P₁, see a catalogue (http://www.sigmaaldrich.com/content/dam/sigma-aldrich/docs/Sigma/General_ Information/nuclease_pl.pdf) of Merck & Co., Inc.).

In a certain embodiment in which the kit or method of the present invention comprises or uses or substantially comprises or comprises uses RNase, the RNase may not contribute to or not substantially contribute to luminescent reaction by luciferase.

In a certain embodiment in which the kit of the present invention substantially comprises RNase, the kit of the present invention may not comprise or not substantially comprise any enzyme that produces ATP from AMP. In a certain embodiment in which the method of the present invention substantially uses RNase, the method of the present invention may not use or not substantially use any enzyme that produces ATP from AMP.

In a certain embodiment in which the kit or method of the present invention comprises or uses RNase, the present invention conducts measurement of the level of luminescence before the RNase completely acts, for example, before ATP derived from RNA has effect on the measurement of ATP, ADP, and AMP that are not derived from RNA. For example, the measurement can be conducted at the time at which the level of luminescence when an RNase is contained is twice or less of, 1.8 times or less of, 1.5 times or less of, 1.2 times or less of, 1.1 times or less of, or equivalent to the level of luminescence when no RNase is contained. The measurement time can be set depending on the amount of the RNase as appropriate and can be, for example, within 10 minutes, within 5 minutes, within 4 minutes, preferably within 3 minutes, within 2 minutes, or within 1 minute, within 30 seconds, or within 10 seconds. Even when a large quantity of RNase is contained, the effect of the RNase can be reduced by shortening the reaction time.

In a certain embodiment in which the kit or method of the present invention comprises or uses RNase, the present invention may be used for a sample containing no RNA or substantially no RNA. Examples of such a sample include samples for which the level of luminescence when an RNase is contained is 2 times or less of, 1.8 times or less of, 1.5 times or less of, 1.2 times or less of, 1.1 times or less of, or equivalent to the level of luminescence when no RNase is contained.

Based on the disclosure herein, various variations are possible. In a certain embodiment, the present invention provides a kit comprising an enzyme that produces AMP from ADP, an enzyme that produces ATP from AMP (for example, PPDK), luciferin, luciferase, and a metal salt and a method for measurement comprising using them. By combining the enzyme that produces AMP from ADP and the enzyme that produces ATP from AMP (for example, PPDK), ADP is converted into AMP and AMP is converted into ATP, and as a result, ATP and ADP and AMP can be measured. This kit may further comprise PEP and PPi. The enzyme that produces ATP from AMP is as described above. Examples of the enzyme that produces AMP from ADP include ADP-dependent hexokinase and apyrase.

### [ADP-Dependent Hexokinase]

The ADP-dependent hexokinase (EC 2.7.1.147) is also referred to as ADP-specific hexokinase and catalyzes the following reaction:

D-glucose+ADP←→D-glucose-6-phosphate+AMP

### [Apyrase]

The apyrase (EC 3.6.1.5) is also referred to as adenosine diphosphatase, ADPase, ATP diphosphatase, or ATP diphosphohydrolase and catalyzes the following two reactions:
ATP+H₂O←→ADP+phosphate (P)
ADP+H₂O←→AMP+phosphate (P)

As used herein, the enzyme that catalyzes a reaction that produces ATP from ADP, PPDK, PWDK, ADK, and the enzyme that produces AMP from ADP described above may be collectively referred to as enzymes having ATP-producing ability.

The enzymes having ATP-producing ability that can be used include any known enzymes such as those derived from microorganisms, bacteria, eukaryotes, protists, plants, and animals, and, for example, a commercially available enzyme can be used. The PPDK is not particularly limited, but examples thereof include those derived from microorganisms such as Microbispora thermorosea described in Patent Literature 4, Propionibacterium shremanii, Bacteroides symbiosus, Entamoeba histolytica, Acetobacter xylinum, and Propionibacter shermanii and those derived from plants such as corn and sugarcane. The ADK is not particularly limited, but examples thereof include those derived from microorganisms such as yeast and those derived from animals such as rabbit, pig, cow, rat, and pig. The PWDK is not particularly limited, but examples thereof include those derived from Escherichia coli, Pseudomonas fluorescens, Pyrococcus furiosus, Staphylothermus marinus, Sulfolobus solfataricus, Thermococcus kodakarensis, Thermoproteus tenax, and corn (Zea mays). The amount of the enzyme added can be set as appropriate depending on the concentration and the reaction system of interest.

Various enzymes having ATP-producing ability are known. As used herein, the activity unit (U) of the enzymes having ATP-producing ability is defined as the amount of the enzyme that converts 1.0 μmole of substrate into ATP per minute at 37° C. at pH 7.8, in view of the ATP-producing ability of the enzymes (1 U=1 μmol ATP/min, pH 7.8, 37° C.). In a certain embodiment, the enzymes having ATP-producing ability can be added such that the activity unit in the system of measurement is 0.001 U or more, 0.01 U or more, 0.1 U or more, 1 U or more, 2 U or more, 3 U or more, 4 U or more, or 5 U or more. In a certain embodiment, the enzymes having ATP-producing ability can be added such that the activity unit in the system of measurement is 10000 U or less, 1000 U or less, 100 U or less, 50 U or less, 10 U or less, 9 U or less, 8 U or less, 7 U or less, or 6 U or less. A person skilled in the art can determine the amount of the enzyme added as appropriate.

When an enzyme having ATP-producing ability is used, the substrate of each enzyme can be added and it is not particularly limited, but, for example, phosphoenolpyruvate and pyrophosphate can be used for PPDK and phosphoenolpyruvate; and acetylphosphate, creatine phosphate, polyphosphate, riboflavin phosphate, and fructose 1,6-bisphosphate can be used for PK, AK, CK, PPK, FMNK, PFK1, and FBPase. For example, phosphoenolpyruvate and phosphate can be used for PWDK. Moreover, for example, glucose can be used for ADP-dependent hexokinase. In a certain embodiment, the kit of the present invention further comprises these substrates. In a certain embodiment, the method of the present invention may further comprise using these substrates.

### [Phosphoenolpyruvate (PEP)]

The method of the present invention may comprise using phosphoenolpyruvate (PEP). The measurement of ATP and AMP present in the system can be promoted by optionally adding an excess amount of PEP to the system. Examples of the concentration of PEP to be used include final concentrations of 0.001 mM to 4500 mM, for example, 2.1 mM.

### [Pyrophosphate (PPi)]

The method of the present invention may comprise using pyrophosphate (PPi). By optionally adding an excess amount of PPi to the system, the measurement of ATP and AMP present in the system can be promoted. Examples of the concentration of PPi to be used include final concentrations of 0.001 mM to 2000 mM, for example, 0.2 mM.

In a certain embodiment, the sample may be treated with a surfactant to lyse cells that may be present. The lysis may lead to the release of intracellular ATP, ADP, or AMP to outside and promote the measurement. The surfactant is not particularly limited, but nonionic surfactants such as tritonX-100, tween-20, tween-80, and brij35; cationic surfactants such as benzalkonium chloride and benzethonium chloride; anionic surfactants such as SDS; and amphoteric surfactants such as CHAPS may be used.

In a certain embodiment, the surfactant does not have adverse effect on the enzyme present in the system or does not significantly reduce the activity thereof. Here, the phrase “not having adverse effect or not significantly reducing the activity thereof” refers to being capable of measuring as a whole without or even with effect. The concentration of the surfactant in the system of measurement may be 0.0001% by weight to 5% by weight, 0.001% by weight to 3% by weight, 0.01% by weight to 2% by weight, 0.1% by weight to 1.5% by weight, or the like.

The reaction reagent may also comprise an enzyme-stabilizing agent such as bovine serum albumin or gelatin that protects a reporter molecule such as luciferase from degradation. A substance that adjusts pH or improves preservation may also be added to the reaction reagent. Examples thereof include appropriate pH buffers (HEPES, Tricine, Tris, phosphate buffer solutions, acetate buffer solutions, and the like), reducing agents (dithiothreitol (DTT), 2-mercaptoethanol, and the like), and sugars (glucose, sucrose, trehalose, and the like).

### [Bio-Related Sample, Bio-Related Instrument]

As used herein, the bio-related sample encompasses any samples to which a biogenic substance may have been attached, wherein ATP contained in the biogenic substance may have been degraded. Moreover, as used herein, the bio-related sample encompasses any bio-related samples that may comprise ATPase. As used herein, the bio-related instrument refers to any instruments to or in which a biogenic substance may be attached or remain, wherein ATP contained in the biogenic substance may have been degraded. Moreover, as used herein, the bio-related instrument encompasses any instruments to or in which ATPase and a human-derived substance may be attached or remain. As used herein, the environment of a bio-related sample or a bio-related instrument refers to, unless otherwise specified, the environment to or in which a biogenic liquid may be attached to and remain, wherein ATP contained in the biogenic liquid may have been degraded. Examples of the environment include, but are not limited to, clothing, protection tools such as gloves, a hand, a finger, a bed, a switch, a doorknob, a bed fence, a nurse call button, a handrail, a washroom, a washbowl, a rest room, and a toilet stool. Samples obtained from such environments shall also be encompassed by the bio-related samples as used herein. The biogenic substance may be derived from a human or an animal. In a certain embodiment, the biogenic substance may be derived from a human. In a certain embodiment, the bio-related sample does not comprise a sample derived from a non-human animal and comprises a sample derived from a human. In a certain embodiment, the bio-related instrument does not comprise a non-human animal-related instrument and comprises a human body-related instrument.

Examples of the biogenic substance include liquids or solids. Examples of the liquids include, but are not limited to, body fluids, blood, lymph, sweat, nasal mucus, tear, saliva, digestive juice, tissue fluid, ascitic fluid, amniotic fluid, spinal fluid, urine, feces, vomiting, and sebum. Examples of the solids include, but are not limited to, solidified liquids, coagulated blood, excrement, scurf, eye mucus, and scab. As used herein, the term “biogenic substance” shall mean, unless otherwise specified, a biogenic substance in which contained ATP may have been degraded. As used herein, the term “biogenic liquid” shall mean, unless otherwise specified, the biogenic liquid in which contained ATP may have been degraded. As used herein, the term “biogenic solid” shall mean, unless otherwise specified, the biogenic solid in which contained ATP may have been degraded. The ATP degradation may be hydrolysis by an enzyme, heat, an agent, acid, alkali, or a combination thereof, or the like.

Examples of the bio-related instrument include medical instruments, and examples thereof include surgical instruments; endoscopes (for example, upper endoscopes to be used for the examination of the esophagus, the stomach, and the duodenum; lower endoscopes to be used in the examination of the rectum and the large intestine; or double balloon small-bowel endoscopes, preferably lower endoscopes); catheters, scalpels, tubes to be inserted in the body of patients, instruments to be inserted in the body of patients, surgical instrument-washing tanks; and medical instrument-washing environments.

In a certain embodiment, the kit of the present invention may comprise an instruction stating that it is for the measurement of cleanliness of a bio-related sample or a bio-related instrument. The instruction may state the method for measurement of cleanliness of a bio-related instrument according to the present invention or the method for using the kit of the present invention.

### [Blood-Related Sample, Blood-Related Instrument]

As used herein, the blood-related sample encompasses any samples to which blood may have been attached. As used herein, the blood-related instrument refers to any instrument to or in which blood may be attached or remain. Examples of the blood-related instrument include medical instruments to or in which blood may be attached or remain. Examples thereof include surgical instruments; endoscopes (for example, upper endoscopes to be used for the examination of the esophagus, the stomach, and the duodenum; lower endoscopes to be used in the examination of the rectum and the large intestine; or double balloon endoscopes, preferably lower endoscopes); catheters, scalpels, tubes to be inserted in the body of patients, instruments to be inserted in the body of patients, surgical instrument-washing tanks; and medical instrument-washing environments. As used herein, the environment of a blood-related sample or a blood-related instrument refers to, unless otherwise specified, the environment to or in which blood may be attached or remain. Examples of the environment include an operating table, a washing tank, clothing, protection tools such as gloves, a hand, a finger, a bed, a handrail, a washroom, a washbowl, and medical facilities. Other examples include sites of accidents, sites of injury cases, and sites where the bloodstain search is performed. Samples obtained from such environments shall be encompassed by the blood-related sample described herein. The blood may be derived from a human or an animal. In a certain embodiment, the blood is derived from a human. In a certain embodiment, the blood does not comprise blood derived from animal meat or fish meat related to food.

Examples of the blood include whole blood, serum, plasma, blood for blood transfusions, collected primary blood, and solutions obtained by diluting primary blood. The blood-related sample also encompasses a solution containing hemocytes (leukocytes, erythrocytes, platelets) or a sample to which the solution may have been attached.

In a certain embodiment, the blood-related sample does not encompass collected blood itself (referred to as the primary sample for convenience). In this embodiment, for example, the “solution containing hemocytes” encompassed in the blood-related sample does not encompass blood itself. In a certain embodiment, the blood-related sample refers to a secondary sample derived from an instrument or an environment that has been contacted with a primary sample. The secondary sample may be obtained by wiping an instrument or an environment that may have been contacted with a primary sample with a cotton swab or the like. In a certain embodiment, the method of the present invention examines whether blood is attached or blood remains to or in a secondary sample. In a certain embodiment, the blood-related sample may be a sample in which blood present therein has been diluted by a washing treatment or the like.

A bio-related instrument or medical instrument to be examined with the kit or by the method of measurement of the present invention is particularly preferably a lower endoscope. This is because the measurement of not only ATP, but also ATP and ADP, ATP and AMP, or ATP, ADP, and AMP is considered to allow accurate detection since the digestive juice present in the intestine contains not only ATP, but also ADP and AMP in a large amount and a lower endoscope is expected to comprise ATP and ADP-degrading enzymes due to the environment in which it is used.

In a certain embodiment, the sample measured by the method of the present invention may be a sample stored at a low temperature, a moderate temperature, or a high temperature such as 0 to 99° C., for example, 0 to 95° C., 4 to 90° C., 4 to 10° C., 10 to 25° C., 25 to 30° C., 30 to 50° C., 37 to 50° C., 50 to 90° C., 60 to 80° C. The sample may be a sample stored at a moderate temperature or a high temperature for a long time, for example, 5 minutes or more, 10 minutes or more, for example, 15 minutes, 20 minutes, 30 minutes, 1 hour, 2 hours, 3 hours, 4 hours, 5 hours, 6 hours, 8 hours, 10 hours, 12 hours, 18 hours, 24 hours, 36 hours, 48 hours, 72 hours, 96 hours, 120 hours, or more.

In a certain embodiment, the sample measured by the method of the present invention may be a sample in which contained ATP has been degraded or may have been degraded into ADP. In a certain embodiment, the sample measured by the method of the present invention may be a sample in which 10 to 60%, 15 to 50%, 18 to 45%, for example, 20 to 40% of the contained ATP has been degraded or may have been degraded into ADP. In a certain embodiment, the sample measured by the method of the present invention may be a sample in which 10 to 60%, 15 to 50%, 18 to 45%, for example, 20 to 40% of the contained ATP has been degraded or may have been degraded into ADP by heating or storage for 2 to 120 hours, for example, 4 to 100 hours, 8 to 96 hours, 16 to 84 hours, 24 to 72 hours. In a certain embodiment, the sample measured by the method of the present invention may be a sample in which 10 to 60%, 15 to 50%, 18 to 45%, for example, 20 to 40% of the contained ATP has been degraded or may have been degraded into ADP by heating or storage at pH 3 to 12, for example, pH 4 to 11. In a certain embodiment, the sample measured by the method of the present invention may be a sample in which 10 to 60%, 15 to 50%, 18 to 45%, for example, 20 to 40% of the contained ATP has been degraded or may have been degraded into ADP by heating or storage at pH 3 to 12, for example, pH 4 to 11 for 2 to 120 hours, for example, 4 to 100 hours, 8 to 96 hours, 16 to 84 hours, 24 to 72 hours.

In a certain embodiment, the kit of the present invention may comprise an instruction stating that it is for the measurement of cleanliness of a blood-related sample or a blood-related instrument. The instruction may state the method for measurement of cleanliness of a blood-related sample or a blood-related instrument according to the present invention or the method for using the kit of the present invention.

In a certain embodiment, the method of the present invention does not comprise step of deactivating ATPase. ATPase present in the blood can be deactivated, for example, by adding trichloroacetic acid (TCA) or trifluoroacetic acid (TFA) to the blood. Since the method of the present invention can also measure ADP and AMP produced by the degradation of ATP, the step of deactivating ATPase is not essential.

### [ATP Assay Method Using Luciferase]

The method of assay using luciferase is described below. The conditions are exemplary. An ATP measurement reagent containing the following is prepared.
1 mM MES
5.1 mM Magnesium acetate
0.15 mM Potassium pyrophosphate
2.1 mM Potassium phosphoenol pyruvate
0.8 mM Luciferin
25 mM Tricine
12.5 μg of luciferase protein/ml (the luciferase concentration (mg protein/mL) is based on the absorbance at 280 nm.)

0.1 mL of a sample solution containing ATP is added to 0.1 mL of the aforementioned ATP measurement reagent, and luminescence is measured. The level of luminescence can be measured using a known luminometer (CentroLB960 or Lumat3 LB9508 from Berthold Technologies GmbH & Co. KG, a luminometer from Kikkoman Biochemifa Company, or the like). The luminescence can be expressed as the relative luminescence unit (RLU) compared to a defined standard. A standard curve is generated using a substrate solution whose ATP concentration is known. Then, the aforementioned ATP measurement reagent is added to a sample solution whose ATP concentration is unknown, and luminescence is measured in the same conditions.

### [ATP+ADP Assay Method 1 (Luciferase+PK)]

A method of ATP+ADP assay is described below. The conditions are exemplary.

An ATP+ADP measurement reagent containing the followings is prepared.
1 mM MES
5.1 mM Magnesium acetate
0.15 mM Potassium pyrophosphate
2.1 mM Potassium phosphoenol pyruvate
0.8 mM Luciferin
25 mM Tricine
12.5 mg of luciferase protein/ml (the luciferase concentration (mg protein/mL) is based on the absorbance at 280 nm.)
25 U/mL PK

### [ATP+ADP Assay Method 2 (Luciferase+AK)]

A method of ATP+ADP assay is described below. The conditions are exemplary.

An ATP+ADP measurement reagent containing the followings is prepared.
1 mM MES
5.1 mM Magnesium acetate
0.15 mM Potassium pyrophosphate
2.1 mM Potassium phosphoenol pyruvate
0.8 mM Luciferin
25 mM Tricine
12.5 μg of luciferase protein/ml (the luciferase concentration (mg protein/mL) is based on the absorbance at 280 nm.)
25 U/mL AK

### [ATP+AMP Assay Method (Luciferase+PPDK)]

A method of ATP+AMP assay is described below. The conditions are exemplary.

An ATP+AMP measurement reagent containing the followings is prepared.
1 mM MES
5.1 mM Magnesium acetate
0.15 mM Potassium pyrophosphate
2.1 mM Potassium phosphoenol pyruvate
0.8 mM Luciferin
25 mM Tricine
12.5 μg of luciferase protein/ml (the luciferase concentration (mg protein/mL) is based on the absorbance at 280 nm.)
2 U/mL PPDK

### [ATP+ADP+AMP Assay Method 1 (Luciferase+PK+PPDK)]

A method of ATP+AMP+ADP assay is described below. The conditions are exemplary.

An ATP, AMP+ADP measurement reagent containing the followings is prepared.
1 mM MES
5.1 mM Magnesium acetate
0.15 mM Potassium pyrophosphate
2.1 mM Potassium phosphoenol pyruvate
0.8 mM Luciferin
25 mM Tricine
12.5 μg of luciferase protein/ml (the luciferase concentration (mg protein/mL) is based on the absorbance at 280 nm.)
25 U/mL PK
2 U/mL PPDK

Alternatively, PWDK instead of PPDK as described above may be used (2 U/mL). In this case, phosphate is used instead of potassium pyrophosphate.

### [ATP+ADP+AMP Assay Method 2 (Luciferase+PK+ADK)]

A method of ATP+AMP+ADP assay is described below. The conditions are exemplary.

An ATP, AMP+ADP measurement reagent containing the followings is prepared.
1 mM MES
5.1 mM Magnesium acetate
0.15 mM Potassium pyrophosphate
2.1 mM Potassium phosphoenol pyruvate
0.8 mM Luciferin
25 mM Tricine
12.5 μg of luciferase protein/ml (the luciferase concentration (mg protein/mL) is based on the absorbance at 280 nm.)
25 U/mL PK
500 U/mL ADK

### [ATP+ADP+AMP Assay Method 3 (Luciferase+PPDK+ADP-Dependent Hexokinase or Apyrase)]

A method of ATP+AMP+ADP assay is described below. The conditions are exemplary.

An ATP, AMP+ADP measurement reagent containing the followings is prepared.
1 mM MES
5.1 mM Magnesium acetate
0.15 mM Potassium pyrophosphate
2.1 mM Potassium phosphoenol pyruvate
0.8 mM Luciferin
25 mM Tricine
12.5 μg of luciferase protein/ml (the luciferase concentration (mg protein/mL) is based on the absorbance at 280 nm.)
30 U/mL ADP-dependent hexokinase +10 mM glucose or 1 U/m apyrase 2 U/mL PPDK

### EXAMPLES

The present invention is more specifically described referring to Examples below. However, the technical scope of the present invention is not limited at all by the Examples.

### Example 1

To examine change of the ATP degradation contained in samples derived from blood over time, a luminescence reagent containing luciferin and luciferase derived from Luciola lateralis (Kikkoman Biochemifa Company, cat. No. 61314) was used. The PK made by Biozyme Laboratories International, Ltd. (cat. No. PK3) was used. The PPDK described in Patent Literature 4 was used. The composition of the luminescence reagent is as follows. The pH of the luminescence reagent was 7.7.

TABLE 1

| | ATP | | ATP + ADP | | ATP + ADP + AMP | |
|---|---|---|---|---|---|---|
| MES | 1 | mM | 1 | mM | 1 | mM |
| Magnesium acetate | 5.1 | mM | 5.1 | mM | 5.1 | mM |
| Potassium pyrophosphate | 0.15 | mM | 0.15 | mM | 0.15 | mM |
| Potassium phosphoenol pyruvate | 2.1 | mM | 2.1 | mM | 2.1 | mM |
| Luciferin | 0.8 | mM | 0.8 | mM | 0.8 | mM |
| Tricine (pH7.7) | 25 | mM | 25 | mM | 25 | mM |
| Luciferase | 12.5 | μg protein/ml | 12.5 | μg protein/ml | 12.5 | μg protein/ml |
| PK | | | 25 | U/mL | 25 | U/mL |
| PPDK | | | | | 2 | U/mL |

The procedure was as follows. First, sheep whole blood was diluted 50 times in purified water (20 μl whole blood+1 mL sterile ultra pure water). Then, this was stored at 25° C., and sampled at 0 minutes, 30 minutes, 60 minutes, and 120 minutes. In the measurement, a 20 times dilute of this (50 μL of sample+950 μL of sterile ultra pure water) was used. 10 μL of the 20 times diluted sample was added to 100 μL of a measurement reagent (the ATP measurement reagent, the ATP+ADP measurement reagent, or the ATP+ADP+AMP measurement reagent), and 35 seconds later, the measurement was started using Lumitester C-110 (Kikkoman Biochemifa Company). The values are the mean of the samples (n=2).

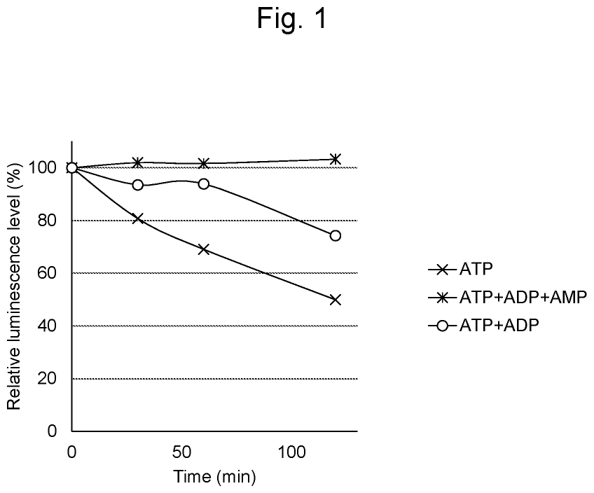

The results are shown in FIG. 1. When measured for only ATP, the level of luminescence greatly decreased over time through 30 minutes, 60 minutes, and 120 minutes. In contrast, the value for ATP and ADP was relatively maintained, and the level of luminescence was stable within 90 to 93% until around 60 minutes. Moreover, when measured for ATP+ADP+AMP, the nucleotides derived from whole blood could be accurately measured even in the sample after 120 minutes.

### Example 2

### Evaluation of ATP Degradation by Heating

In brief, an ATP solution at pH 4, 7, or 11 was heated at 80° C. for a predetermined time. ATP, ATP+AMP or ATP+AMP+ADP contained in samples after heating were measured. The amount of ATP+ADP was determined by subtracting the value of (ATP+AMP) from the value of (ATP+AMP+ADP) and adding the value of ATP.

The procedure was as follows. First, the following buffer solution was prepared:
0.05 mol phthalic acid (pH 4.0)
0.05 mol phosphoric acid (pH 6.9)
0.05 mol glycocoll, 0.05 mol sodium chloride, 0.05 mol sodium hydroxide (pH 11.3)

Then, samples for heating were prepared:
ATP solution (1 mM) 0.05 mL
Each buffer solution 10 mL
Final concentration 5×10⁻³ mM

These samples were subdivided and stored at 80° C., and sampled at each time point. The samples were then cryopreserved until the measurement. Then, each ATP solution was diluted 100 times in sterile ultra pure water for the measurement:
0.99 mL sterile ultra pure water
0.01 mL ATP solution

This was measured using Lumitester C-110 (Kikkoman Biochemifa Company). The number of samples was n=2:
0.1 mL luminescence reagent (the ATP measurement reagent, the ATP+AMP measurement reagent, or the ATP+ADP+AMP measurement reagent)
0.01 mL ATP solutions stored at various pH for each duration

The composition of the luminescence reagents is as follows.

TABLE 2

| | ATP | | ATP + AMP | | ATP + ADP + AMP | |
|---|---|---|---|---|---|---|
| MES | 1 | mM | 1 | mM | 1 | mM |
| Magnesium acetate | 5.1 | mM | 5.1 | mM | 5.1 | mM |
| Potassium pyrophosphate | 0.15 | mM | 0.15 | mM | 0.15 | mM |
| Potassium phosphoenol pyruvate | 2.1 | mM | 2.1 | mM | 2.1 | mM |
| Luciferin | 0.8 | mM | 0.8 | mM | 0.8 | mM |
| Tricine (pH7.7) | 25 | mM | 25 | mM | 25 | mM |
| Luciferase | 12.5 | μg protein/ml | 12.5 | μg protein/ml | 12.5 | μg protein/ml |
| PK | | | | | 25 | U/mL |
| PPDK | | | 2 | U/mL | 2 | U/mL |

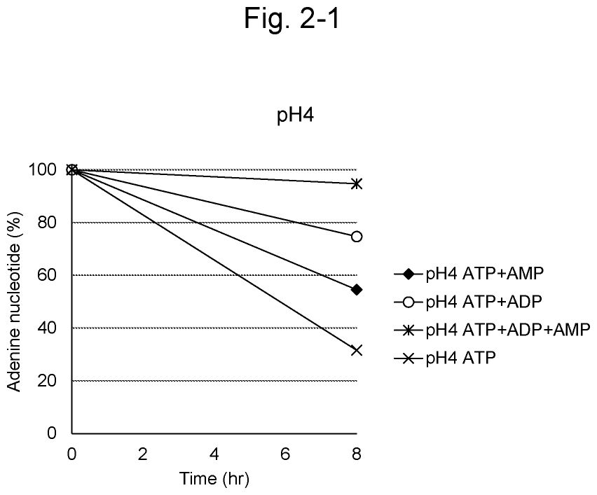

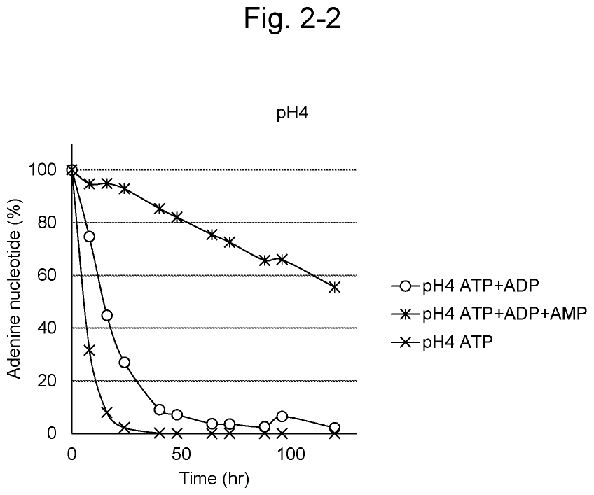

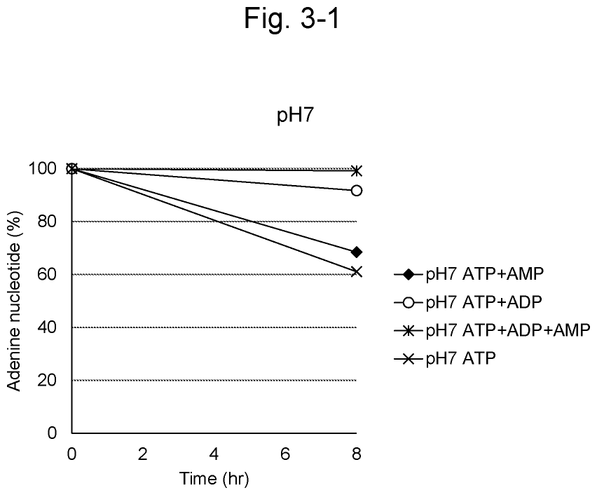

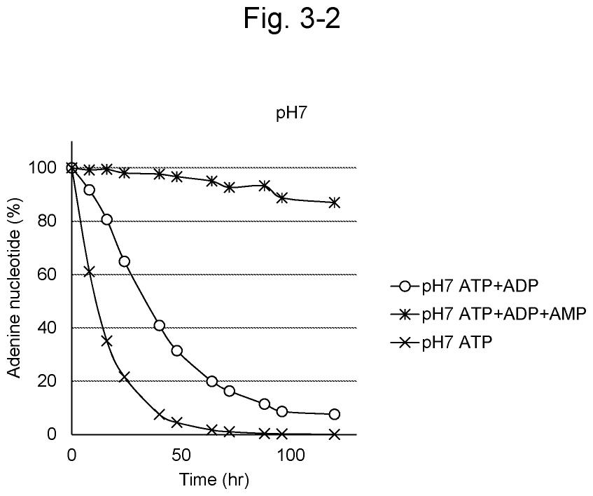

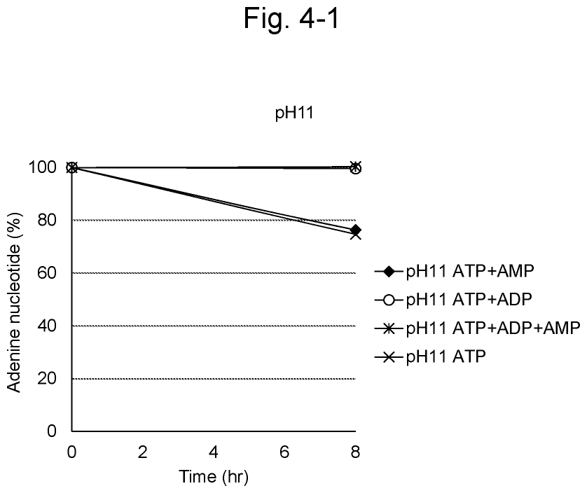

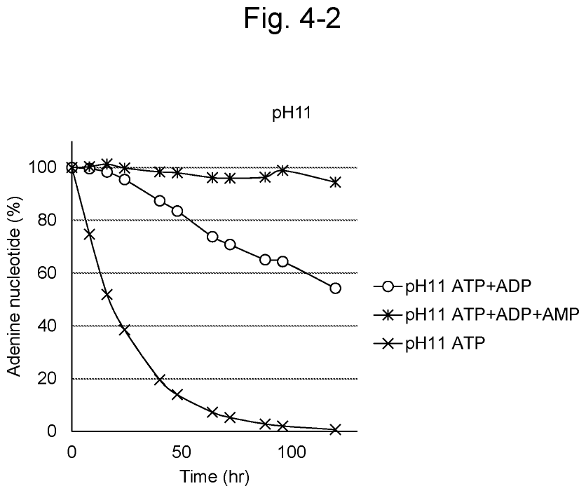

The results are shown in FIGS. 2-1 to 4-2. It was found that when measured for only ATP, the accurate measurement is difficult because of rapid ATP degradation by heating. Meanwhile, it was found that more accurate measurement is possible when measured for ATP and ADP. Furthermore, it was found that more accurate measurement is possible when measured for ATP, AMP, and ADP.

### ATP and ADP Measurement of Heated Sample

The amount of ATP+ADP contained in the samples can also be measured using luciferase and PK.

The procedure is as follows. First, the following buffer solution is prepared:
0.05 mol phthalic acid (pH 4.0)
0.05 mol phosphoric acid (pH 6.9)
0.05 mol glycocholate, 0.05 mol sodium chloride, 0.05 mol sodium hydroxide (pH 11.3)

Then, samples for heating are prepared:
ATP solution (1 mM) 0.05 mL
Each buffer solution 10 mL
Final concentration 5×10⁻³ mM

These samples are subdivided and stored at 80° C., and sampled at each time point. The samples are then cryopreserved until the measurement. Then, each ATP solution is diluted 100 times in sterile ultra pure water for the measurement:
0.99 mL sterile ultra pure water
0.01 mL ATP solution

This is measured using Lumitester C-110 (Kikkoman Biochemifa Company).
0.1 mL luminescence reagent (the ATP+ADP measurement reagent)
0.01 mL ATP solutions stored at various pH for each duration

A standard curve for ATP+ADP can be generated beforehand from standard samples with known concentrations.

For example, standard samples of various concentrations of ATP or ADP (1×10⁻⁹ M to 1×10⁻⁶ M) were prepared and measured using the luminescence reagent available for measuring ATP described in Example 1, to which PK was added at 25 U/ml and using Lumitester C-110 (Kikkoman Biochemifa Company) (N=3).
0.1 ml of luminescence reagent (the ATP+ADP measurement reagent)
0.01 ml ATP or ADP solutions at various concentrations

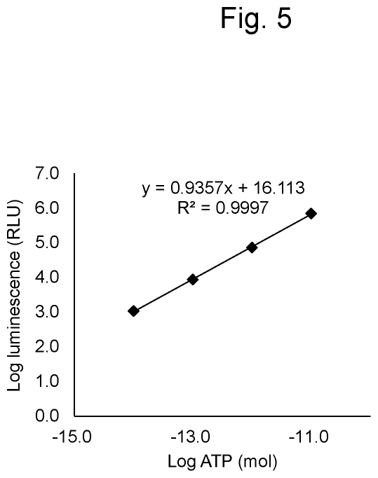

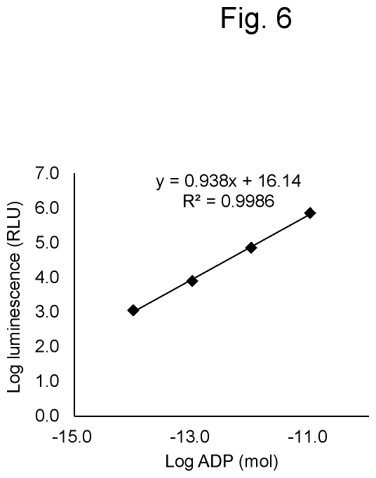

The mol amounts of ATP and ADP in the solutions emitting luminescence were calculated, and a standard curve was generated. The results are illustrated in FIGS. 5 and 6.

### Example 3

### Change of ATP Degradation in Saliva Over Time

Assuming that a sample containing ATP to be measured is contaminated with saliva, saliva recovered using Saliva Collection Aid (SALIMETRICS) was added to a 0.2 μM ATP solution at 200 times dilution to prepare a saliva sample.

This was mixed with the luminescence reagent at the following ratios, and the mixture was measured using Lumitester C-110 (Kikkoman Biochemifa Company). The number of samples was n=2:
0.1 mL luminescence reagent (the ATP measurement reagent, the ATP+AMP measurement reagent, or the ATP+ADP+AMP measurement reagent)
0.01 mL saliva sample

The composition of the luminescence reagents is as set forth in Table 2 in Example 2.

The samples were stored at 25° C., and the level of luminescence was measured 60, 120, 210, and 330 minutes after the addition of ATP. The relative level of luminescence was calculated, when the level of luminescence 60 minutes later is 100%.

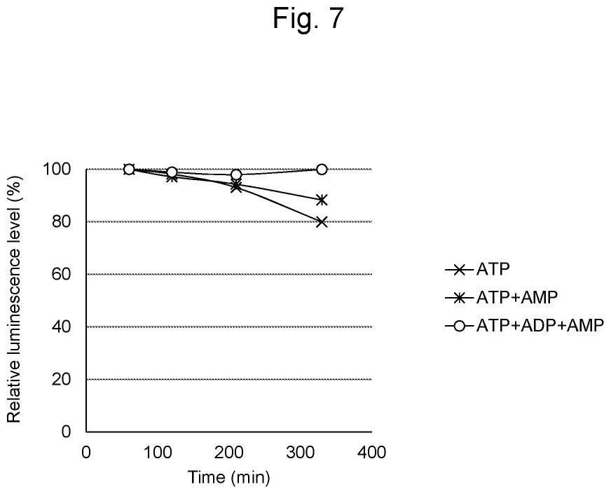

The result is shown in FIG. 7. It was found that when measured for only ATP, the accurate measurement is difficult because of ATP degradation. Meanwhile, it was found that more accurate measurement is possible when measured for ATP and AMP and that further accurate measurement is possible when measured for ATP, AMP, and ADP.

### Example 4

### Change of ATP Degradation on Endoscope Over Time

A lower endoscope (manufactured by Olympus Corporation, used) was wiped with a 40 cm cotton swab LuciSwab 3.2-400 (manufactured by Kikkoman Biochemifa Company). The wipe was suspended in a 5% glucose solution, which was diluted 4 times in sterile ultra pure water to prepare an endoscope sample. The sample was mixed with the luminescence reagent at the following ratios, and the mixtures were measured using Lumitester C-110 (Kikkoman Biochemifa Company).
0.1 mL luminescence reagent (the ATP measurement reagent, the ATP+AMP measurement reagent, or the ATP+ADP+AMP measurement reagent)
0.01 mL endoscope sample

The composition of the luminescence reagents is as set forth in Table 2 in Example 2.

The endoscope sample was stored at 25° C., and the luminescence was measured just after storage and at 0.5 and 1 hour. The relative level of luminescence was calculated, when the level of luminescence just after storage is 100%.

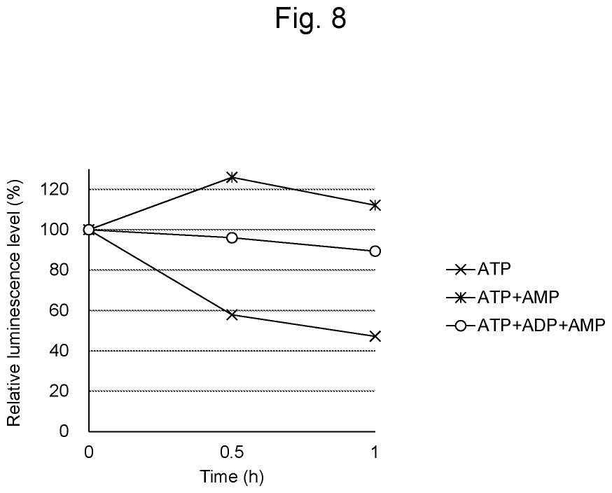

The result is shown in FIG. 8. It was found that when measured for only ATP, the accurate measurement is difficult because of ATP degradation. When measured for ATP and AMP, the level of luminescence increased greatly. This is considered to be because the sample originally contained a large amount of ADP and this is degraded into AMP to increase the amount of ATP+AMP. From this result, it was found that the accurate measurement is difficult when measured for ATP and AMP. Meanwhile, it was found that accurate measurement is possible with small change of the value over time when measured for ATP, AMP, and ADP.

### Example 5

Construction of System of Measurement for ATP+ADP+AMP Using Enzyme that Catalyzes Reaction that Produces AMP from ADP

ADP-dependent hexokinase (ASAHI KASEI PHARMA CORPORATION, T-93 ADP-HKTII), which is an enzyme that catalyzes a reaction that produces AMP from ADP, and glucose were added to the luminescence reagent for ATP+AMP measurement in Table 2 in Example 2, and the mixture was examined whether the measurement of ATP+ADP+AMP was possible.

The composition of the luminescence reagent is as follows.

TABLE 3

| | |
|---|---|
| MES | 1 mM |
| Magnesium acetate | 5.1 mM |
| Potassium pyrophosphate | 0.15 mM |
| Potassium phosphoenol pyruvate | 2.1 mM |
| Luciferin | 0.8 mM |
| Tricine (pH7.7) | 25 mM |
| Luciferase | 12.5 μg protein/ml |
| Glucose | 10 mM |
| Hexokinase | 30 U/mL |
| PPDK | 2 U/mL |

Standard samples of various concentrations of ATP, ADP, or AMP (1×10⁻⁹ M to 1×10⁻⁶ M) were prepared and mixed with the aforementioned luminescence reagent at the following ratios. This was measured using Lumitester C-110 (Kikkoman Biochemifa Company) (n=2):
0.1 ml Luminescence reagent
0.01 ml Various concentrations of ATP, ADP, or AMP solution

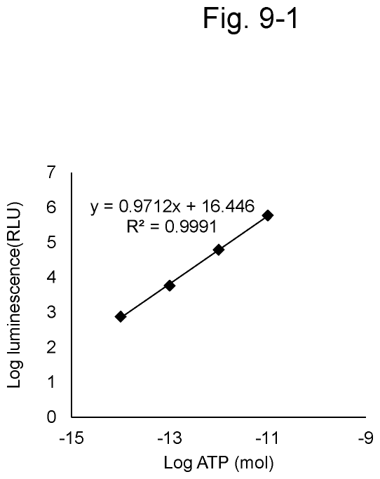

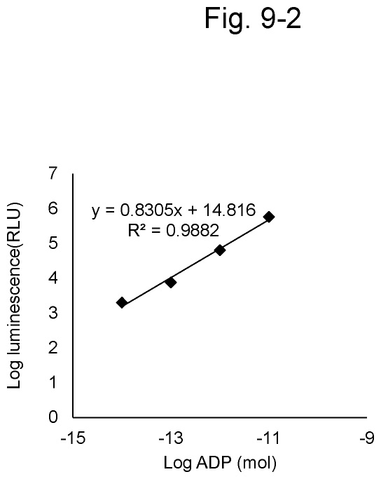

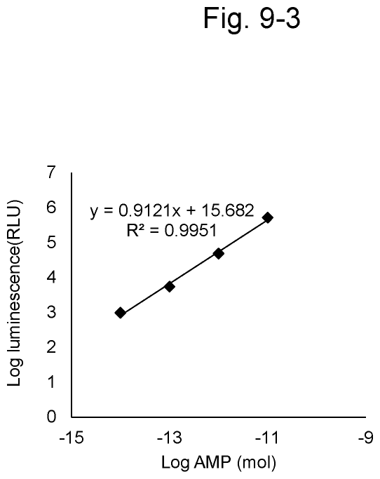

The mol amounts of ATP, ADP, and AMP in the solutions emitting luminescence were calculated, and standard curves were generated. The results are shown in FIGS. 9-1 to 9-3.

Subsequently, apyrase (Sigma A6536), which is an enzyme that catalyzes a reaction that produces AMP from ADP, was added to the luminescence reagent for ATP+AMP measurement in Table 2 in Example 2, and the mixture was examined whether the measurement of ATP+ADP+AMP was possible as described above.

The composition of the luminescence reagent is as follows.

TABLE 4

| | |
|---|---|
| MES | 1 mM |
| Magnesium acetate | 5.1 mM |
| Potassium pyrophosphate | 0.15 mM |
| Potassium phosphoenol pyruvate | 2.1 mM |
| Luciferin | 0.8 mM |
| Tricine (pH7.7) | 25 mM |
| Luciferase | 12.5 μg protein/ml |
| Apyrase | 1 U/mL |
| PPDK | 2 U/mL |

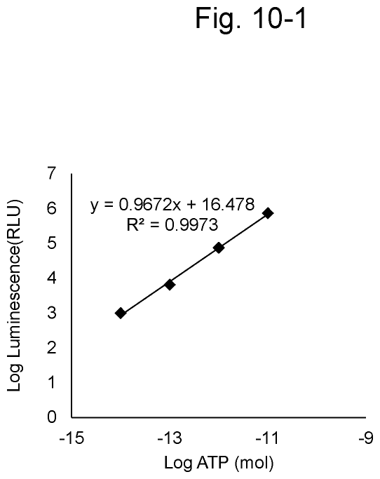

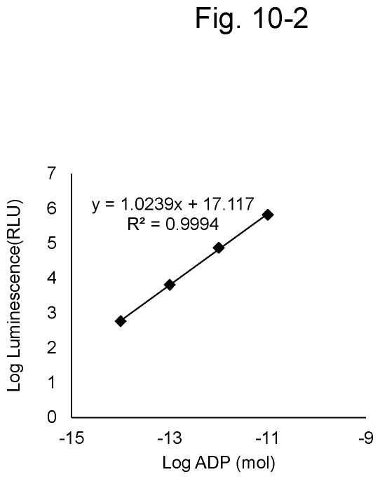

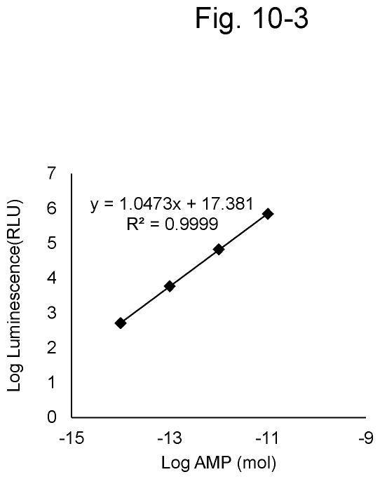

The mol amounts of ATP, ADP, and AMP in the solutions emitting luminescence were calculated, and standard curves were generated. The results are shown in FIGS. 10-1 to 10-3.

The results indicate that the measurement of ATP+ADP+AMP is possible by using a combination of PPDK, which is an enzyme that catalyzes a reaction that produces ATP from AMP, and an enzyme that catalyzes a reaction that produces AMP from ADP.

### INDUSTRIAL APPLICABILITY

According to the present invention, the cleanliness of blood-related samples or blood-related instruments can be measured. Moreover, blood adhered or remaining to or in instruments can be detected.

All publications, patents, and patent applications cited herein shall be incorporated herein by reference as they are.

1. A kit for measuring cleanliness of a blood-related sample or a blood-related instrument, comprising an enzyme that catalyzes a reaction that produces ATP from ADP, luciferin, luciferase and a metal salt.

2. A kit for measuring cleanliness of a bio-related sample or a bio-related instrument, comprising an enzyme that catalyzes a reaction that produces ATP from ADP, luciferin, luciferase, and a metal salt.

3. The kit according to claim 1, wherein the enzyme that catalyzes a reaction that produces ATP from ADP is selected from the group consisting of pyruvate kinase (PK), acetate kinase (AK), creatine kinase (CK), polyphosphate kinase (PPK), hexokinase, glucokinase, glycerol kinase, fructokinase, phosphofructokinase, riboflavin kinase, and fructose-bisphosphatase.

4. The kit according to claim 1, further comprising an enzyme that catalyzes a reaction that produces ADP or ATP from AMP.

5. The kit according to claim 4, wherein the enzyme that catalyzes a reaction that produces ADP or ATP from AMP is pyruvate orthophosphate dikinase (PPDK), adenylate kinase (ADK), or pyruvate-water dikinase (PWDK).

6-8. (canceled)

9. The kit according to claim 1, wherein the blood-related sample is a sample to or in which blood may be attached or remain, or the blood-related instrument is an instrument to or in which blood may be attached or remain.

10. The kit according to claim 2, wherein the bio-related sample is a sample to or in which a biogenic substance may be attached or remain, wherein ATP contained in the biogenic substance may have been degraded, or the bio-related instrument is an instrument to or in which a biogenic substance may be attached or remain, wherein ATP contained in the biogenic substance may have been degraded.

11-12. (canceled)

13. A method for measuring cleanliness of a blood-related sample or a blood-related instrument, comprising using an enzyme that catalyzes a reaction that produces ATP from ADP, luciferin, luciferase, and a metal salt.

14. A method for measuring cleanliness of a bio-related sample or a bio-related instrument, comprising using an enzyme that catalyzes a reaction that produces ATP from ADP, luciferin, luciferase, and a metal salt.

15. The method according to claim 13, wherein the enzyme that catalyzes a reaction that produces ATP from ADP is selected from the group consisting of pyruvate kinase (PK), acetate kinase (AK), creatine kinase (CK), polyphosphate kinase (PPK), hexokinase, glucokinase, glycerol kinase, fructokinase, phosphofructokinase, riboflavin kinase, and fructose-bisphosphatase.

16. The method according to claim 13, further comprising an enzyme that catalyzes a reaction that produces ADP or ATP from AMP.

17. The method according to claim 16, wherein the enzyme that catalyzes a reaction that produces ADP or ATP from AMP is pyruvate orthophosphate dikinase (PPDK), adenylate kinase (ADK), or pyruvate-water dikinase (PWDK).

18-20. (canceled)

21. The method according to claim 13, wherein the blood-related sample is a sample to or in which blood may be attached or remain, or the blood-related instrument is an instrument to or in which blood may be attached or remain.

22. The method according to claim 14, wherein the bio-related sample is a sample to or in which a biogenic substance may be attached or remain, wherein ATP contained in the biogenic substance may have been degraded, or the bio-related instrument is an instrument to or in which a biogenic substance may be attached or remain, wherein ATP contained in the biogenic substance may have been degraded.

23-24. (canceled)
